(19) United States
(12) Patent Application Publication
(10) Pub. No.: US 2016/0176919 A1
(43) Pub. Date: Jun. 23, 2016

(54) CELL SPECIFIC LABELING OF NEWLY SYNTHESIZED PROTEINS

(71) Applicants: Michael Cohen, Portland, OR (US); Ruth Barrett, Portland, OR (US); Haihong Jin, Portland, OR (US)

(72) Inventors: Michael Cohen, Portland, OR (US); Ruth Barrett, Portland, OR (US); Haihong Jin, Portland, OR (US)

(73) Assignee: OREGON HEALTH & SCIENCE UNIVERSITY, Portland, OR (US)

(21) Appl. No.: 14/978,700

(22) Filed: Dec. 22, 2015

Related U.S. Application Data
(60) Provisional application No. 62/096,364, filed on Dec. 23, 2014.

(51) Int. Cl. C07K 1/13; C07H 19/16; C12N 9/84; A01K 67/027; C12N 15/85

(52) U.S. Cl. CPC C07K 1/13; A01K 67/0275; C12N 15/8509; C12N 9/84; C07H 19/16; A01K 2227/105; C12Y 305/01011; A01K 2217/206

(57) ABSTRACT

Disclosed herein are compositions comprising blocked puromycin analogs that are converted into active puromycin analogs upon the activity of a penicillin acylase. Also disclosed are methods of using blocked puromycin analogs to label proteins in a selected cell type in vivo in a transgenic multicellular organism that expresses a penicillin acylase within the selected cell type. Also disclosed are transgenic mice expressing a penicillin acylase within a selected cell type.

### FIELD

In general, the field is the labeling of nascent proteins. More specifically, the field is the labeling of nascent proteins in particular cell types in vivo.

### BACKGROUND

Most cells within an organism have the same genome, but the mRNA transcripts and proteins that are actually expressed at any point in the life of the cell vary widely. The sets of transcripts (which can also be referred to as the transcriptome) and proteins (which can also be referred to as the proteome) expressed by a cell are more indicative of cellular identity (Wang Z et al, Nat Rev Genet 10, 57 (2009); incorporated by reference herein). In addition, the transcriptome and proteome of a specific cell can change depending on the physiological or pathophysiological context, for example, during different stages of development, in response to various stimuli, or in disease, including in different stages of disease (Kong J and Lasko P, Nat Rev Genet 13, 383 (2012); incorporated by reference herein). Methods of profiling the transcriptome and proteome of distinct cell populations in multicellular organisms are therefore important to understand the dynamics of regulation of transcriptional and translational processes in physiology and pathophysiology.

Technologies such as microarrays and whole exome sequencing, allow facile profiling of many RNA transcripts simultaneously. These have accelerated the understanding of transcriptional regulation (Wang et al, 2009 supra). However, one major obstacle to more detailed transcriptome and proteome profiling in a cell-specific, temporally-resolved manner has been the difficulty in purifying homogenous cell populations from heterogeneous tissues. Cells in vivo are under the control of paracrine, juxtacrine, hormonal, and other influences, therefore, even the very act of isolating a cell from its environment can alter the cell's transcriptome and proteome, which in turn results in inaccuracy with regard to the true transcriptome and proteome the cell uses under a particular set of conditions.

One example of this phenomenon is the pancreatic islet. Pancreatic alpha cells clearly affect the activity of pancreatic beta cells and vice versa (Yang Y P et al, Genes Dev 25, 1680 (2011); incorporated by reference herein). Several strategies for cell-specific RNA isolation in pancreatic cells have been developed (Heiman M et al, Cell 135, 738 (2008); Doyle J P et al, Cell 135, 749 (2008); and Miller M R et al, Nat Methods 6, 439 (2009); all of which are incorporated by reference herein). One such method, “TU-tagging,” uses the Toxoplasma gondii enzyme, uracil phosphoribosyltransferase (UPRT), to convert 4-thiouracil (4-TU) into TU-monophosphate, which is then incorporated into nascent RNA transcripts (Miller et al, 2009 supra). Cells that have been engineered to express the UPRT enzyme then generate TU-tagged RNA, which can then be biotinylated and purified. Another approach, Translating Ribosome Affinity Purification (TRAP), is based on the cell-specific immunopurification of epitope tagged ribosomes associated with actively translated mRNAs (Heiman et al 2008, supra and Doyle et al, 2008 supra). While these systems, particularly TRAP, can enrich for protein-coding mRNAs that are in the process of being translated, neither one actually monitors protein synthesis. In fact, another study demonstrated that noncoding RNAs are enriched using TRAP, by demonstrating that not all RNAs bound to the ribosome are undergoing translation (Zhou P et al, Proc Nati Acad Sci USA 110, 15395 (2013); incorporated by reference herein).

Furthermore, the act of translation of mRNA into protein is regulated through multiple posttranscriptional mechanisms such as cap dependent initiation, polyadenylation, and microRNA repression (Kong J and Lasko P, Nat Rev Genet 13, 383 (2012); incorporated by reference herein). Translational regulation allows cells to respond rapidly to various stimuli, such as electrical activity and metabolic changes. A stimulus can result in global increases in protein synthesis, augmentation of groups of proteins within a particular pathway, or induction of specific proteins. In addition to this temporal control of gene expression, translational regulation can also control the spatial expression of genes in various subcellular compartments such as the nucleus or mitochondria. A variety of physiologically important events are mediated by translational regulation including skeletal muscle hypertrophy (Goodman C A et al, FASEB J 25, 1028 (2011); incorporated by reference herein), memory consolidation (Costa-Mattioli M et al, Neuron 61, 10 (2009); incorporated by reference herein), and glucose signaling (Gomez E et al, J Biol Chem 279, 53937 (2004); incorporated by reference herein), as discussed further herein. All of these signify the importance of monitoring the proteome rather than just the transcriptome.

One method of metabolic labeling of proteins has been developed using E. coli cells. The method involves the use of an E. coli strain that express a mutant methionyl-tRNA synthetase, that allows incorporation of the methionine bioisostere, azidonorleucine into proteins expressed in the E. coli strain (Ngo J T et al, Nat Chem Biol 5, 715 (2009); incorporated by reference herein). Non mutant E. coli cells that do not express the mutant methionyl-tRNA synthetase do not incorporate azidonorleurcine. While this strategy is useful for cell-specific proteome labeling in E. coli where the amount of methionine can be controlled, it is unlikely to work in multicellular organisms because it would be difficult to remove all methionine from such organisms. Additionally, overexpression of a mutant methionyl-tRNA synthetase or replacement of the endogenous methionyl-tRNA synthetase, necessarily resulting in the replacement of methionine for azidonorleurcine in complex organisms is likely to disrupt normal protein function.

A widely used method for identifying newly synthesized proteins is metabolic labeling with the azidohomoalanine (Aha), a methionine bioisostere analog. Following incorporation of Aha into nascent polypeptides during translation, Aha-labeled proteins can be conjugated to an alkyne tag via a copper(I) catalyzed [3+2] cycloaddtion, commonly referred to as the “click reaction” (Dieterich D C et al, Nat Protoc 2, 532 (2007); incorporated by reference herein). Using the click reaction, Aha can be conjugated to a biotin alkyne tag then affinity purified using methods involving, for example, avidin-agarose beads or on-bead trypsin digestion. Mass spectrometry of the affinity purified biotinylated protein results in a snapshot of the nascent proteome. While this method can be used in complex organisms, it labels all nascent proteins in all cells and therefore does not allow for identifying newly synthesized protein in a particular cell or tissue type. Methods and compositions that can be used in labeling a nascent proteome in complex organisms are needed.

### SUMMARY

Disclosed herein are compounds of the formula:

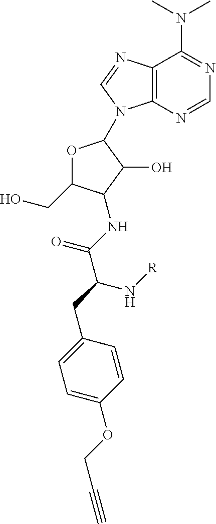

wherein R is acyl. One particular example of the compound is a compound of the formula:

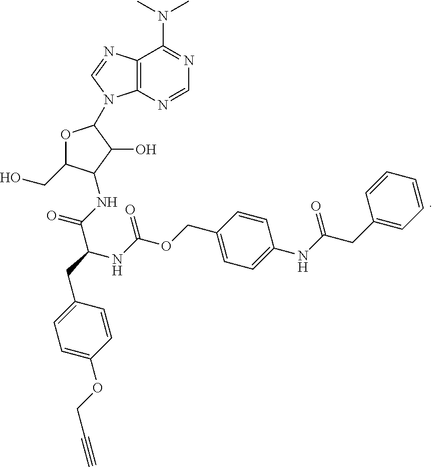 [SMILES: C#CCOc1ccc(C[C@H](NC(=O)OCc2ccc(NC(=O)Cc3ccccc3)cc2)C(=O)NC2C(CO)OC(n3cnc4c(N(C)C)ncnc43)C2O)cc1]

Also disclosed are methods of generating an isolated set of proteins from a subject. The method involves administering a blocked puromycin analog to the subject. The blocked puromycin analog can be converted into an active puromycin analog by a penicillin acylase. The subject can be any transgenic multicellular organism that expresses the penicillin acylase in a selected cell type. The active puromycin analog is conjugated to the set of proteins during translation. A sample that includes cells of the selected cell type is collected from the subject. The set of proteins is then purified from the sample on the basis of having incorporated the active puromycin analog. Examples of blocked puromycin analogs include the compounds disclosed above. If the compounds disclosed above constitute the blocked puromycin analog, then the active puromycin analog is O-propargyl puromycin and the acylase is a penicillin G acylase. In further examples of the method, the set of proteins is labeled by conjugated a label to the conjugated puromycin analog. The label can be any label, including a fluorophore or biotin. In some examples, the subject expresses a penicillin G acylase with expression driven by a constitutively active promoter. The construct also comprises a loxP-flanked stop cassette that prevents expression of the penicillin G acylase. The subject also expresses cre recombinase with expression driven by a conditionally active promoter.

Disclosed herein are transgenic mice that express a penicillin G acylase in particular cells. Examples of such mice include mice with an expression construct that includes: penicillin G acylase and a stop cassette that prevents the expression of the penicillin G acylase. The stop cassette is flanked by loxP sites. This construct also has a constitutively active promoter operably linked to the penicillin G acylase/stop cassette. The mouse also has an expression construct that includes a conditional promoter (such as a cell or tissue specific promoter) operably linked to cre recombinase. In such mice, cre is expressed in a cell of interest thereby activating expression of penicillin G acylase in the cell of interest.

It is an object of the invention to provide a system by which the proteome of a particular cell type can be analyzed in context within a complex organism.

### BRIEF DESCRIPTION OF THE SEVERAL VIEWS OF THE DRAWINGS

FIG. 1 is a cartoon depicting the mechanism of action of puromycin. Puromycin binds to a translating ribosome and is subsequently added to a nascent polypeptide via its alpha-amino group. At low concentrations, puromycin is selectively added to the C-terminus of the polypeptide.

FIG. 2 is a set of chemical structures depicting the activity of penicillin G acylase on PhAc-OP-puromycin to convert it to OP-puromycin. PhAc-OP-puro comprises a PhAc blocking group and a spacer, the spacer being linked to OP-puro via a carbamate linkage. The blocking group is removed by PGA and the spacer spontaneously fragments via sequential 1,6-quinone methide rearrangement and decarboxylation to yield OP-puro.

FIG. 3 is a set of two images of gels that show selective enrichment of newly synthesized proteins in Hela cells treated with OP-puro relative to those treated with puromycin. Hela cells were treated with either 50 μM OP-puro or puro (as indicated) for one hour. Cells were then harvested in PBS+1% SDS (sodium dodecyl sulfate) and lysed. Lysates were diluted in 0.1% SDS and incubated with biotin azide using a click reaction for one hour. Biotinylated proteins were affinity purified using NeutrAvidin agarose beads. Eluted proteins were resolved using a 4-12% gradient SDS-PAGE gel and detected by either Western blot with streptavidin-HRP (left gel) or by silver stain (right gel). The lanes reflect input (I), supernatant (S), or pull-down (P) as indicated.

FIG. 4A is a dot blot and a bar graph showing HEK293T cells transfected with either a PGA IRES GFP or a control plasmid (as indicated) using calcium phosphate transfection. Twenty-four hours after transfection, cells were treated with 5 or 50 μM of DMSO (negative control), OP-puro, or PhAc-OP-puro as indicated for 30 minutes. Cells were harvested in PBS+1% SDS. Lysates were diluted 10× and incubated with biotin azide under click reaction conditions for one hour. Lysates were spotted onto nitrocellulose and biotinylated proteins detected with Streptavidin-HRP (top panel). Dot intensity was quantified using Image) and graphed (bottom panel).

FIG. 4B is a set of two images of gels showing proteins from three of the samples described in FIG. 4A resolved by 4-12% gradient SDS-PAGE and detected by Western blot with Streptavidin-HRP (left panel) or Ponceau S staining (right panel) Bands marked with an asterisk are endogenous cellular biotinylated proteins.

FIG. 5A is a set of 12 images of HEK 293T cells transfected with PGA-IRES-GFP or controls as indicated using calcium phosphate transfection. In two of the conditions, PGA-IRES-GFP transfected cells were mixed with nontransfected cells such that the total population of transfected cells was 5%. At a timepoint 24 hours post transfection, cells were treated with either a DMSO control or 5 μM PhAc-OP-puro for 30 minutes as indicated. Cells were then fixed with methanol and nascent protein synthesis detected by click chemistry with Alexa Fluor-568 azide. After click chemistry, cells were labeled with an antibody specific for GFP. Total cells were detected by DAPI.

FIG. 5B is a set of four images of E18 rat hippocampal neurons transfected with PGA IRES GFP using lipofectamine 2000 and otherwise treated as described in FIG. 5A.

FIG. 6 is a plot of the results of incubating PhAc-OP-puro in mouse plasma for the indicated period of time. Stability of PhAc-OP-puro was monitored by liquid chromatography mass spectrometry.

### SEQUENCE LISTING

SEQ ID NO: 1 is a sequence of E. coli penicillin G acylase.

SEQ ID NO: 2 is a sequence of Achromobacter xylosoxidans penicillin G acylase.

SEQ ID NO: 3 is a sequence of Alcaligenes faecalis penicillin G acylase.

SEQ ID NO: 4 is a sequence of Bacillis badius penicillin G acylase.

### DETAILED DESCRIPTION

Terms:

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” In addition, the materials, methods, and examples are illustrative only and not intended to be limiting. In order to facilitate review of the various embodiments of the disclosure, the following explanations of specific terms are provided:

Administration: To provide or give a subject an agent, such as a puromycin derivative, by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Acyl: An acyl group is a chemical group with the structure X₁—CO—X₂. Acyl groups include aldehydes, which have the structure X₁—CO—H, esters, which have the structure X₁—COO—X₂, and amides, which have the structure X₁—CO—NX₂X₃ wherein X₁, X₂, and X₃ can be H or any organic group such as an alkyl or aryl group.

Alkyl: a branched or unbranched saturated hydrocarbon group, such as, without limitation, methyl, ethyl, n-propyl, isopropyl, n-butyl, isobutyl, t-butyl, pentyl, hexyl, heptyl, octyl, nonyl, decyl, tetradecyl, hexadecyl, eicosyl, tetracosyl and the like. A lower alkyl group is a saturated branched or unbranched hydrocarbon having from 1 to 6 carbon atoms (C₁₋₆alkyl). The term alkyl also encompasses cycloalkyls. Alkyl also encompasses substituted alkyls which are alkyl groups wherein one or more hydrogen atoms are replaced with a substituent such as, without limitation, alkyl, alkynyl, alkenyl, aryl, halide, nitro, amino, ester, ether, ketone, aldehyde, hydroxyl, carboxyl, cyano, amido, haloalkyl, haloalkoxy, or alkoxy. The term alkyl also encompasses heteroalkyls. A heteroalkyl contains at least one heteroatom such as nitrogen, oxygen, sulfur, or phosphorus replacing one or more of the carbons. Substituted heteroalkyls are also encompassed by the term alkyl.

Aryl: any carbon-based aromatic group including, but not limited to, benzyl, naphthyl, and phenyl. The term aryl also contemplates substituted aryls in which one or more of the hydrogens is substituted with one or more groups including but not limited to alkyl, alkynyl, alkenyl, aryl, halide, nitro, amino, ester, ether, ketone, aldehyde, hydroxy, carboxylic acid, cyano, amido, haloalkyl, haloalkoxy, or alkoxy. The term aryl also contemplates heteroaryls in which one or more of the carbons is replaced by a heteroatom. Examples of heteroatoms include, but are not limited to, nitrogen, oxygen, sulfur, and phosphorous. Substituted heteroaryls are also encompassed by the term aryl.

Cre recombinase: an enzyme that catalyzes recombination between two loxP sites. LoxP sites are 34-base sequences comprising an 8-base core sequence (5′-GCATACAT-3′) where recombination takes place flanked by two 13 base inverted repeats (5′-ATAACTTCGTATA-3′) and (5′-TATACGAAGTTAT-3′). Depending on the orientation of the LoxP sites, the Cre recombinase reaction can result in an inversion, translocation, or deletion of the sequence flanked by the loxP sites. Deletion occurs when the loxP sites are oriented in the same direction on the same chromosome flanking the sequence to be deleted. A sequence of DNA flanked by loxP sequences can be referred to as ‘floxed.’ The cre recombinase can be expressed in a transgenic organism and expressed using a conditionally active promoter to achieve expression in a particular cell type.

Floxed-STOP cassette: An engineered mutation that allows for the repair of genes in the presence of cre recombinase. LoxP sites flank a DNA spacer that prevents the expression of a gene of interest (for example, a penicillin acylase.) The spacer can be inserted into any part of the gene, for example, in the first intron. In the presence of cre recombinase, the fragment is excised and the expression of the gene of interest proceeds. The fragment can comprise one or more stop codons and/or selective marker genes (Sauer B L and Petyuk V A, US 2006/0014264 (2006); incorporated by reference herein.)

Label: A label can be any substance capable of aiding a machine, detector, sensor, device, column, or enhanced or unenhanced human eye from differentiating a labeled composition from an unlabeled composition. Labels may be used for any of a number of purposes and one skilled in the art will understand how to match the proper label with the proper purpose. Examples of uses of labels include purification of biomolecules, identification of biomolecules, detection of the presence of biomolecules, detection of protein folding, and localization of biomolecules within a cell, tissue, or organism. Examples of labels include but are not limited to: radioactive isotopes (such as carbon-14 (¹⁴C)) or chelates thereof; dyes (fluorescent or nonfluorescent), stains, enzymes, nonradioactive metals, magnets, protein tags, small molecules, haptens, either half of a receptor/ligand pair, any antibody epitope, any specific example of any of these; any combination between any of these, or any label now known or yet to be disclosed. A label may be covalently attached to a biomolecule or bound through hydrogen bonding, Van Der Waals or other forces. A label may be covalently or otherwise bound to the N-terminus, the C-terminus or any amino acid of a polypeptide.

One particular example of a label is a protein tag. A protein tag comprises a sequence of one or more amino acids that may be used as a label as discussed above, particularly for use in protein purification. In some examples, the protein tag is covalently bound to the polypeptide. It may be covalently bound to the N-terminal amino acid of a polypeptide, the C-terminal amino acid of a polypeptide or any other amino acid of the polypeptide. Often, the protein tag is encoded by a polynucleotide sequence that is immediately 5′ of a nucleic acid sequence coding for the polypeptide such that the protein tag is in the same reading frame as the nucleic acid sequence encoding the polypeptide. Protein tags may be used for all of the same purposes as labels listed above and are well known in the art. Examples of protein tags include chitin binding protein (CBP), maltose binding protein (MBP), glutathione-S-transferase (GST), poly-histidine (His), thioredoxin (TRX), FLAG®, V5, c-Myc, HA-tag, and so forth.

Another particular example of a label is biotin. Biotin is a naturally occurring compound that is an enzyme cofactor with a number of effects in the body. Biotin is also used as a protein label due to its small size, which generally does not affect protein structure or activity. In addition, biotin binds to streptavidin or avidin with very high affinity and is therefore very easily captured by streptavidin/avidin conjugated columns, beads, plates, etc. A number of methods well known in the art have adapted the biotin/(strept)avidin interaction for purification of biotinylated proteins.

Multicellular Organism: Any animal, plant, fungal, or other organism comprising cells of more than one type or function. The cells can be organized into tissues or organs.

Operably Linked: A first nucleic acid sequence is operably linked with a second nucleic acid sequence when the first nucleic acid sequence is placed in such a way that it has an effect upon the second nucleic acid sequence. For instance, a promoter is operably linked to a coding sequence if the promoter affects the transcription or expression of the coding sequence. Operably linked DNA sequences may be contiguous, or they may operate at a distance.

Penicillin acylase: a member of a group of enzymes that catalyze the cleavage of the acyl chain of penicillins to yield 6-amino penicillanic acid. Penicillin acylases are produced by bacteria, actinomycetes, yeasts, and fungi. Penicillin acylases can be further classified into penicillin G acylases, penicillin V acylases and ampicillin acylases on the basis of substrate specificity. One of skill in the art would be able to adapt any currently published penicillin acylase into the methods and transgenic organisms disclosed herein and to test it for its ability to convert a blocked puromycin analog into an active puromycin analog as described herein.

Polypeptide: Any chain of amino acids, regardless of length or posttranslational modification (such as glycosylation, methylation, ubiquitination, phosphorylation, or the like). Herein as well as in the art, the term ‘polypeptide’ is used interchangeably with peptide or protein, and is used to refer to a polymer of amino acid residues. The term ‘residue’ can be used to refer to an amino acid or amino acid mimetic incorporated in a polypeptide by an amide bond or amide bond mimetic. Polypeptide sequences are generally written with the N-terminal amino acid on the left and the C-terminal amino acid to the right of the sequence.

Promoter: A promoter can be any of a number of nucleic acid control sequences that directs transcription of a nucleic acid. Typically, a eukaryotic promoter includes necessary nucleic acid sequences near the start site of transcription, such as, in the case of a polymerase II type promoter, a TATA element or any other specific DNA sequence that is recognized by one or more transcription factors. Expression by a promoter may be further modulated by enhancer or repressor elements. Numerous examples of promoters are available and well known to those of skill in the art. A nucleic acid comprising a promoter operably linked to a nucleic acid sequence that codes for a particular polypeptide can be termed an expression vector. An expression vector comprising a constitutively active promoter expresses the protein at effectively all times in the cell. A conditionally active promoter directs expression only under certain conditions. For example, a conditionally active promoter might direct expression only in the presence or absence of a particular compound such as a small molecule, amino acid, nutrient, or other compound while a constitutively active promoter directs expression independently of such conditions. A conditionally active promoter might direct expression in a particular cell or tissue type such as neurons or neural tissue, pancreatic cells such as pancreatic beta cells, fibroblasts, tumors, etc.

Puromycin analog: refers to a compound having the general aminonucleoside core structure of puromycin. An active puromycin analog is capable of conjugating to the C-terminus of a polypeptide and is modified to include a reactive group capable of undergoing a bioorthogonal reaction. One example of an active puromycin analog is O-propargyl puromycin. Other examples are described in Salic et al, US Pat Publ Number 2013/0122535 (2013); which is incorporated by reference herein. A blocked puromycin analog is a puromycin analog that further comprises a group, such as an acyl group, that renders the puromycin analog incapable of conjugating to the C-terminus of the polypeptide unless it has been acted upon by an enzyme such as a penicillin acylase enzyme.

Sample: A sample, such as a biological sample, is obtained from a subject. As used herein, samples include all samples comprising cells from transgenic organisms in which PGA is expressed and therefore in which a set of proteins can be labeled with a puromycin derivative that is converted to OP-puro by PGA as described herein. Cells that express PGA are selected during the generation of the transgenic organism as described herein.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage identity or similarity (which takes into account conservative amino acid substitutions); the higher the percentage, the more similar the sequences are. Polypeptides or protein domains thereof that have a significant amount of sequence identity and also function the same or similarly to one another (for example, proteins that serve the same functions in different species or mutant forms of a protein that do not change the function of the protein or the magnitude thereof) can be called “homologs.”

Methods of alignment of sequences for comparison are well known in the art. Various programs and alignment algorithms are described in: Smith & Waterman, Adv Appl Math 2, 482 (1981); Needleman & Wunsch, J Mol Biol 48, 443 (1970); Pearson & Lipman, Proc Natl Acad Sci USA 85, 2444 (1988); Higgins & Sharp, Gene 73, 237-244 (1988); Higgins & Sharp, CABIOS 5, 151-153 (1989); Corpet et al, Nuc Acids Res 16, 10881-10890 (1988); Huang et al, Computer App Biosci 8, 155-165 (1992); and Pearson et al, Meth Mol Bio 24, 307-331 (1994). In addition, Altschul et al, J Mol Biol 215, 403-410 (1990), presents a detailed consideration of sequence alignment methods and homology calculations.

The NCBI Basic Local Alignment Search Tool (BLAST) (Altschul et al, (1990) supra) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr database, swissprot database, and patented sequences database. Queries searched with the blastn program are filtered with DUST (Hancock & Armstrong, Comput Appl Biosci 10, 67-70 (1994.) Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 50%, 60%, 70%, 80%, 90%, 95%, 98%, or 99% sequence identity to a nucleic acid that encodes a protein.

Transgene: An exogenous (heterologous) nucleic acid sequence introduced into the genome of an organism that does not normally have the nucleic acid sequence as part of its genome. Examples include nucleic acids encoding PGA or cre recombinase inserted into the genome of a multicellular organism as well as lox sequences, viral promoters, and the like.

Transgenic organism: A multicellular organism (such as an animal, plant or multicellular fungus) having a transgene present as an extrachromosomal element in a portion of its cells or stably integrated into its germline DNA (i.e., in the genomic sequence of most or all of its cells). The transgene is introduced into the germ line of such transgenic animals by genetic manipulation of, for example, embryos or embryonic stem cells of the host animal according to methods well known in the art. The transgene may be introduced in the form of an expression construct that encodes and expresses a particular protein, such as PGA or the cre recombinase. Such expression constructs can also comprise promoter, enhancer and/or suppressor sequences.

### Puromycin and Puromycin Derivatives:

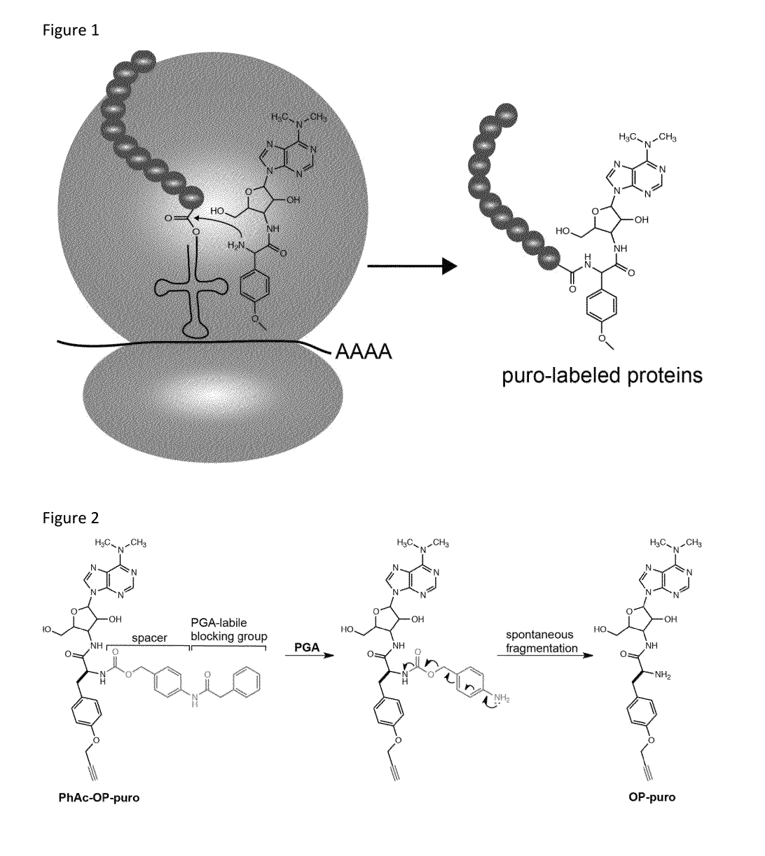

Disclosed herein is a strategy that identifies newly synthesized proteins in vivo in a cell type of interest. This strategy exploits the mechanism of action of the antibiotic puromycin (also referred to herein as ‘puro’). Puro is an aminonucleoside that contains an N,N-dimethyl adenosine fused to an O-methyl-L-tyrosine amino acid, thereby mimicking an amino acid charged tRNA. During active translation, puro binds to the translating ribosome where its α-amino group covalently attacks the carbonyl of the aminoacyl-tRNA ester, causing premature termination of translation (FIG. 1). When used at sufficiently low concentrations, puro selectively incorporates into the C-terminus of full-length proteins during translation without inhibiting translation or inducing a stress response (Schmidt E K et al, Nat Methods 6, 275 (2009); incorporated by reference herein). Thus, the use of low concentrations of puro or puro variants provides a method of labeling and identifying newly synthesized proteins.

Fluorescent derivatives of puro have been used to monitor the rate of protein synthesis as well as to investigate the sub-cellular location of protein synthesis in cells (Smith W B et al, Neuron 45, 765 (2005) and Starck S R et al, Chem Biol 11, 999 (2004); both of which are incorporated by reference herein). In another method, an antibody that specifically binds puro-labeled polypeptides was developed and used to monitor protein synthesis by Western blotting and fluorescence activated cell sorting (FACS).

A variant of puro in which the O-methyl was replaced with an O-propargyl group (also referred to as OP-puro herein) was synthesized and used to visualize nascent protein synthesis in cells after conjugation of the OP-puro-labeled polypeptides to a fluorescent azide tag using click chemistry (Liu J et al, Proc Nati Acad Sci USA 109, 413 (2012) and Salic et al, US Pat Publ Number 2013/0122535 (2013); both of which are incorporated by reference herein). Conjugation of OP-puro-labeled polypeptides to a biotin azide tag through, for example, the click reaction allowed the enrichment and purification of OP-puro-labeled polypeptides using streptavidin beads. It has also been shown that OP-puro can be used to monitor protein synthesis in mice. OP-puro, as described herein, is an example of an active puromycin analog.

While in principle the OP-puro method could be used to identify the newly synthesized proteins in specific cell populations in vivo, this could only be achieved if the desired cells are purified to homogeneity. This would be a daunting challenge, however, especially in heterogeneous tissues, like the brain, and in low abundance cell populations such as stem cells. In addition, as described above, the process of cell purification itself could alter the nascent proteome. This would make the results of such a study difficult to interpret.

Disclosed herein are methods and compositions that solve the problem of identifying newly synthesized proteins in specific cell populations in vivo. The disclosed methods and compositions allow control of the cell-specific action of OP-puro in multicellular organisms, limiting it to selected cell types. Disclosed herein are compounds termed blocked puromycin analogs. Such analogs contain a blocking group, rendering them inactive.

The blocked puromycin analog can be converted into an active puromycin analog when it is acted upon by an enzyme that can remove the blocking group from the blocked puromycin analog. Limiting the expression to a selected cell type of a complex organism results in a system and a method of labeling newly transcribed polypeptides in a selected cell type in a complex organism in vivo. Such a system would provide unprecedented detail in understanding changes in a proteome upon the response to a stimulus or upon contracting a disease.

The disclosed method circumvents the need for laborious cell purification. Because the active puromycin analog is limited to cells that express the enzyme, tissues from the complex organism can simply be homogenized and the labeled proteins purified by any appropriate method, including avidin affinity chromatography after conjugation to biotin using click chemistry.

Puromycin is produced by Streptomyces alboniger (S. alboniger). Most of the enzymes involved in the biosynthesis of puro in S. alboniger have been well studied (Lacalle R A et al, EMBO J 11, 785 (1992); incorporated by reference herein). One enzyme that has received much attention is puromycin N-acetyltransferase (PAC), which adds the acetyl group from acetyl coenzyme A to the α-amino group of puro (Vara J et al, Biochemistry 24, 8074 (1985); incorporated by reference herein). Because the α-amino group is essential for puro function, acylation of the α-amino group acts as a blocking group, thereby rendering puro inactive. PAC, when expressed in mammalian cells, confers resistance to puro (Vara J A et al, Nucl Acids Res 14, 4617 (1986); incorporated by reference herein). Consequently, PAC has been used as a selectable marker that will select for stably transformed mammalian cell lines.

One example of a blocked puromycin analog is an analog of OP-puro in which the α-amine is modified with an enzyme-labile, blocking group. Expression of an enzyme capable of removing this blocking group within a cell that has been treated with the blocked puromycin analog would result in the conversion of the blocked puromycin analog to the activated puromycin derivative OP-puro within the cell. One example of such a blocked puromycin analog is as follows:

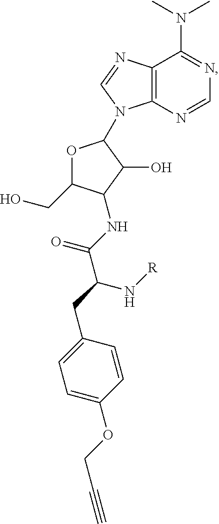

wherein R is acyl.

The OP-puro within the cell would in turn label polypeptides within the cell. The enzyme can be expressed in a cell-specific manner using genetic targeting with the result that OP-puro-labeled proteins would derive only from the targeted cells. Tissue containing the genetically targeted cells can be lysed and the OP-puro-labeled proteins can be tagged with, for example, biotin using the click reaction with biotin azide. Purification and enrichment of the biotin-tagged nascent proteins using avidin-coated beads followed by trypsin digestion and tandem mass spectrometry (MS/MS) analysis can be used to identify newly synthesized proteins in specific cell populations in multicellular organisms such as animals.

The use of enzyme-labile blocking groups for amines has been used in synthetic organic chemistry (Kadereit D and Waldmann H, Chem Rev 101, 3367 (2001); incorporated by reference herein). A blocking group for amines that has received particular attention is the phenylacetyl (PhAc) group, which can be removed by the enzyme penicillin G acylase (PGA). PGA is expressed in E. coli where its function is to remove the PhAc group from the β-lactam antibiotic, penicillin G, generating 6-aminopenicillanic acid (6-APA) (Volpato G et al, Curr Med Chem 17, 3855 (2010); incorporated by reference herein). PGA is exquisitely selective for amides of phenylacetic acid and does not hydrolyze peptide bonds. Analysis of the crystal structure of PGA bound to a penicillin G derivative shows that the PhAc moiety binds to a specificity pocket in the active site ensuring selective cleavage of PhAc (McVey C E et al, J Mol Biol 313, 139 (2001); incorporated by reference herein). PGA is a stable and robust enzyme that can be made in bacteria in high yield. The most widespread application of PGA to date is for the industrial production of semi-synthetic penicillin-like compounds, such as amoxicillin, which are prepared from 6-APA (Volpato G et al, Curr Med Chem 17, 3855 (2010); incorporated by reference herein). In addition to its natural substrate penicillin G, PGA can remove the PhAc group from diverse compounds, including amino acids, nucleotides (Waldmann H R, Agnew Chem Int Ed Engl 36, 647 (1997); incorporated by reference herein), and fluorescent reporters (Ninkovic M et al, Anal Biochem 292, 228 (2001); incorporated by reference herein), in high yields.

PhAc-OP-puro (compound 3)

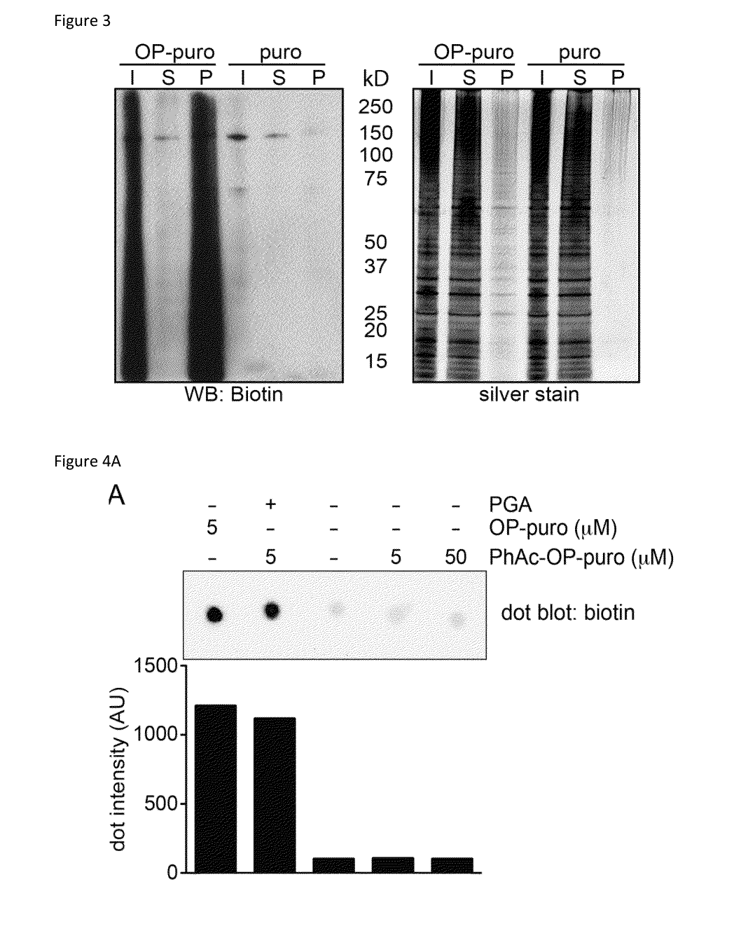

PhAc-OP-puro (compound 3) is an analog of OP-puro that comprises a PhAc blocking group that blocks puro activity, but can be cleaved by PGA and a stable N-(benzyloxy) carbamate spacer that can undergo a spontaneous fragmentation upon cleavage of the PhAc group by PGA to generate OP-puro (FIG. 2).

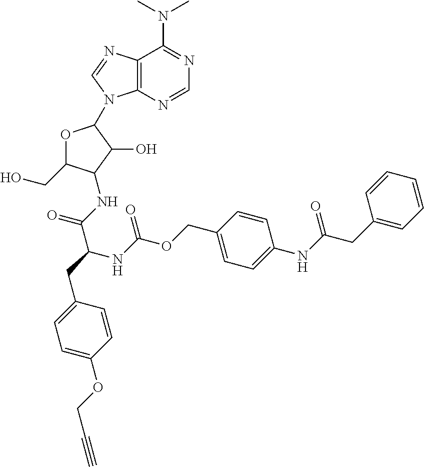 [SMILES: C#CCOc1ccc(C[C@H](NC(=O)OCc2ccc(NC(=O)Cc3ccccc3)cc2)C(=O)NC2C(CO)OC(n3cnc4c(N(C)C)ncnc43)C2O)cc1]

PhAc-OP-puro was synthesized in one step by reacting the α-amine group of OP-puro (synthesized as described in Liu et al, 2012 supra) with a 4-nitrophenyl carbonate-activated spacer of compound 2. Synthesis of the spacer+blocking group of compound 2 is shown in Scheme 1 below.

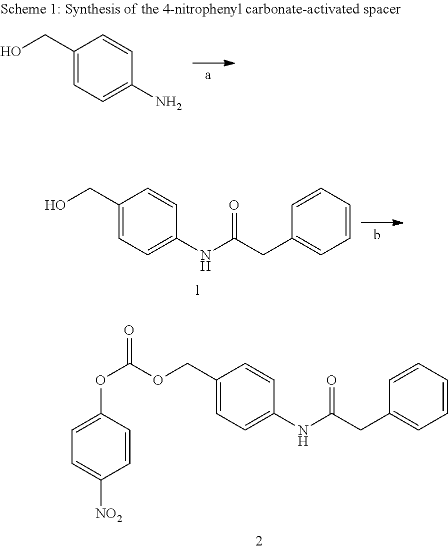 [SMILES: Nc1ccc(CO)cc1 ; O=C(Cc1ccccc1)Nc1ccc(CO)cc1 ; O=C(Cc1ccccc1)Nc1ccc(COC(=O)Oc2ccc([N+](=O)[O-])cc2)cc1]

The particular spacer was selected because it would minimize the potential steric hindrance between PhAc-OP-puro and PGA. As shown in FIG. 2, when the blocking group is removed by PGA, the spacer undergoes spontaneous fragmentation to yield OP-puro. The PGA-labile, spacer-based blocking group for amines has been used successfully for the synthesis of peptides (Kadereit D and Waldmann H, Chem Rev 101, 3367 (2001); incorporated by reference herein).

PhAc-OP-puro should be inert in wild type mammalian cells because the α-amine group of OP-puro is blocked. As described above, the blocking group of PhAc-OP-puro can be removed in mammalian cells expressing active PGA, thus revealing the α-amine group of OP-puro, allowing it to incorporate into nascent polypeptides. In this way, new protein synthesis can be monitored, and newly synthesized proteins identified with labeling limited to PGA expressing cells.

In some examples, the Cre-loxP recombination system to can be used to genetically target the expression of PGA to specific cell populations and regulate gene expression (Nagaraj N et al, Mol Syst Biol 7, 548 (2011); incorporated by reference herein.) This system is a well-known technique for the generation of cell-type specific, transgene expression in mice. One example of the use of this system includes an expression module comprising the PGA gene downstream of a floxed STOP, which prevents transcription of PGA. When bred to mice that express Cre in a cell-specific manner, the progeny will lack the STOP sequence in tissues that express Cre recombinase, resulting in the desired pattern and timing of PGA expression in specific cell populations of interest.

### EXAMPLES

The following examples are illustrative of disclosed methods. In light of this disclosure, those of skill in the art will recognize that variations of these examples and other examples of the disclosed method would be possible without undue experimentation.

### Example 1

### Identifying the Nascent Proteome in Hela Cells Using OP-Puro

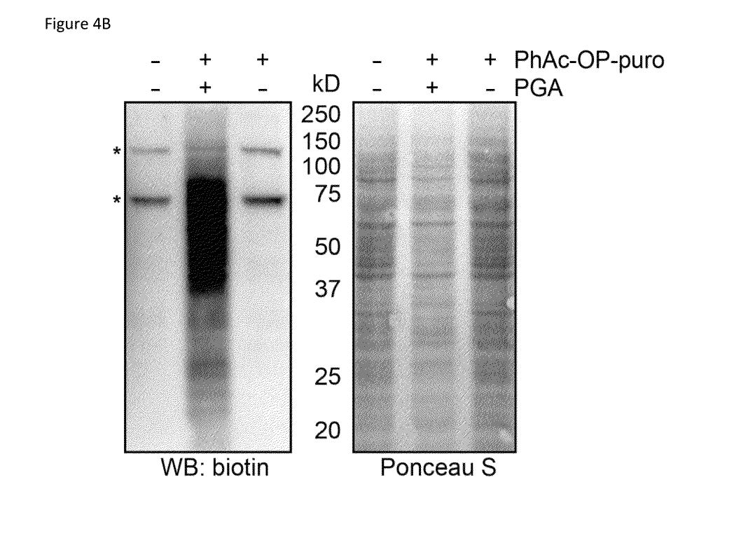

OP-puro can identify newly synthesized proteins in a defined timeframe in mammalian cells grown in culture. Newly synthesized proteins were identified in Hela cells using OP-puro, followed by click chemistry, affinity purification, and MS/MS analysis. OP-puro was synthesized according to Liu et al, 2012 supra. Western blot analysis of the NeutrAvidin eluates revealed the presence of biotinylated proteins only in eluates from OP-treated cells (FIG. 3). Silver stain analysis demonstrated minimal, nonspecific binding to the NeutrAvidin agarose beads, with no detectable staining in eluates from puro-treated cells (FIG. 3).

To prepare samples for MS/MS analysis, the captured biotinylated proteins from the OP-puro and puro (used as a background control) treated cells were alkylated and digested on the agarose beads using trypsin. The peptides were analyzed using a capillary HPLC system coupled online with an LTQ orbitrap mass spectrometer. Comparison of the two treatment conditions revealed 1,600 peptides with unique amino acid sequences from OP-puro treated Hela cells. These peptides identified 530 different human proteins, which represents ˜5% of the total Hela proteome (Nagaraj et al 2011 supra). Together, these results demonstrate that OP-puro coupled with click chemistry, affinity chromatography, and MS/MS analysis can be used to identify newly synthesized proteins in cells.

### Example 2

### Efficient Deblocking of PhAc-OP-Puro by PGA in Mammalian Cells

In E. coli, PGA is expressed as a precursor protein containing a signal sequence that targets it to the bacterial periplasm, where it is processed and activated (Kasche et al, Biochem Biophys Acta 1433, 76 (1999); incorporated by reference herein). Previous studies, however, have shown that by removing the signal sequence, PGA can be expressed in the cytoplasm of the methylotrophic yeast, Pichia pastoris (Maresova H et al, BMC Biotechnol 10, 7 (2010); incorporated by reference herein). Importantly, this cytoplasmic variant of PGA was as active as wild type (WT) PGA expressed in bacteria (Maresova H et al, 2010 supra). However, to date, no one had shown whether or not PGA could be expressed in mammalian cells or any cells derived from a multicellular organism. A construct (PGA-IRES-GFP) was the designed, the construct comprising a variant of PGA lacking the signal sequence described above, and an IRES-GFP for co-cistronic expression of GFP to identify transfected cells.

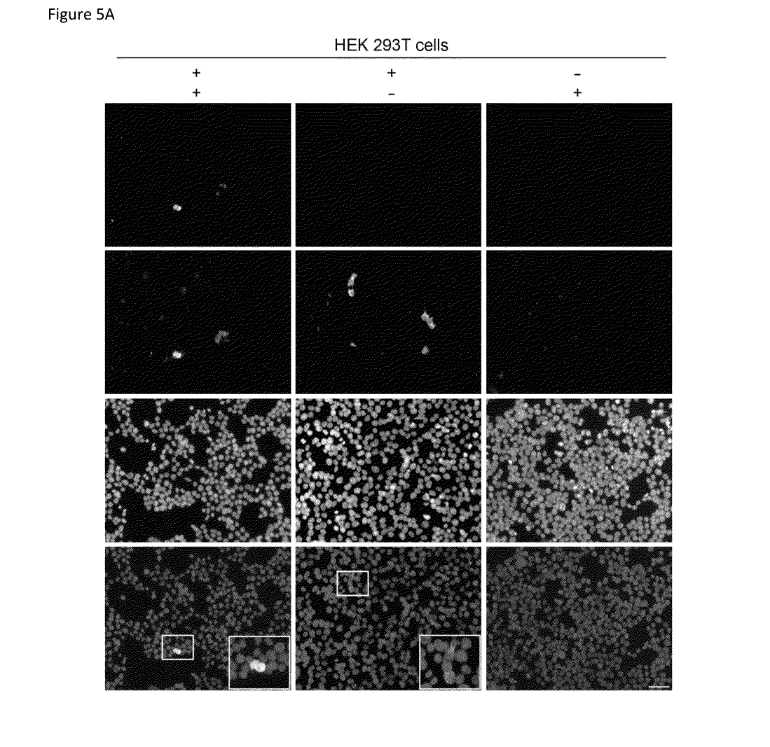

The incorporation of OP-puro into newly synthesized proteins was detected using click chemistry with biotin azide. The results show that in human embryonic kidney (HEK) 293T cells transfected with the PGA-IRES-GFP construct, PhAc-OP-puro (5 μM, 30 min) was converted to OP-puro, which could then incorporate into newly synthesized proteins as indicated by the prominent biotin signal (FIG. 4A and FIG. 4B). The signal intensity was the same as that of control transfected cells treated with OP-puro (5 μM). This suggests that the PhAc-OP-puro used to treat the cells is completely converted to OP-puro within 30 min. (FIG. 4A). Under these conditions there was no observed induction of the unfolded protein response. By contrast, PhAc-OP-puro (up to 50 μM) was completely inert in the absence of PGA (FIG. 4A and FIG. 4B). These results show that in mammalian cells, PGA can efficiently deblock PhAc-OP-puro, which is inert in cells not expressing PGA, to generate OP-puro.

### Example 3

### Cell-Specific Labeling of Newly Synthesized Proteins

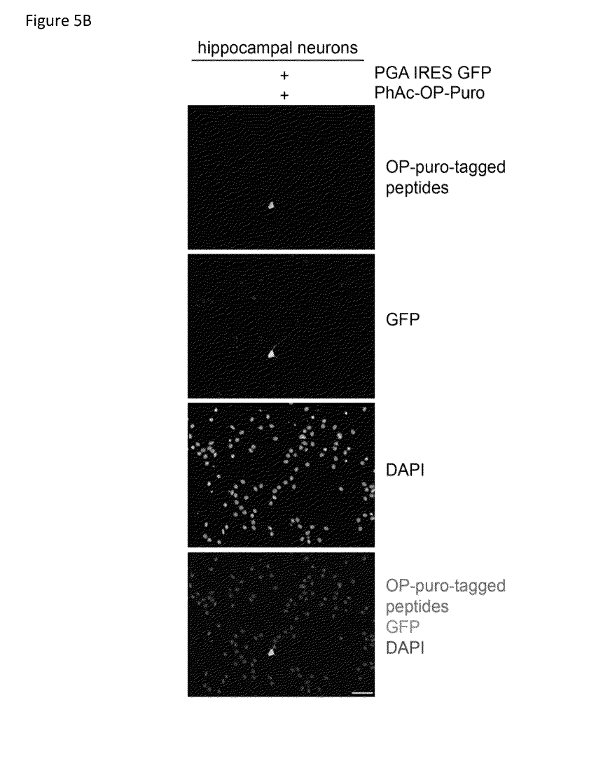

To further demonstrate that the deblocking of PhAc-OP-puro occurs only in cells expressing PGA, the incorporation of OP-puro into newly synthesized proteins in single cells was monitored using click chemistry with Alexa Fluor-568 azide. In cells treated with PhAc-OP-puro (5 μM, 30 min), labeling of newly synthesized proteins (OP-puro-tagged peptides) is detected in PGA-expressing cells (identified by GFP) (FIG. 5A). The amount of labeling appeared to correlate with the expression levels of PGA as determined by comparing the fluorescence intensity of the OP-puro tagged peptides to the fluorescence intensity of GFP. No labeling was detected in cells not expressing PGA (FIG. 5A). Similar results were obtained in neurons, demonstrating that our approach is applicable to other cell types (FIG. 5B). These results show that cell-specific labeling of newly synthesized proteins can be achieved in PGA expressing cells treated with PhAc-OP-puro.

### Example 4

### PhAc-OP-Puro is Stable in Mouse Plasma

As shown above, PhAc-OP-puro is stable in cell culture and is not converted to OP-puro in wild type cells. To use PhAc-OP-puro in an animal system such as mice, the in vitro stability of PhAc-OP-puro in plasma should be determined. Plasma typically contains high esterase activity that could result in cleavage of the carbamate linkage in PhAc-OP-puro. Cleavage of PhAc-OP-puro at the carbamate linkage would generate OP-puro, and therefore would not allow use of the disclosed method in intact mice. While there are several examples of drugs containing carbamates (Chaturvedi D, Tetrahedron 68, 15 (2012); incorporated by reference herein), it is unpredictable whether or not a particular carbamate is resistant to esterases in plasma.

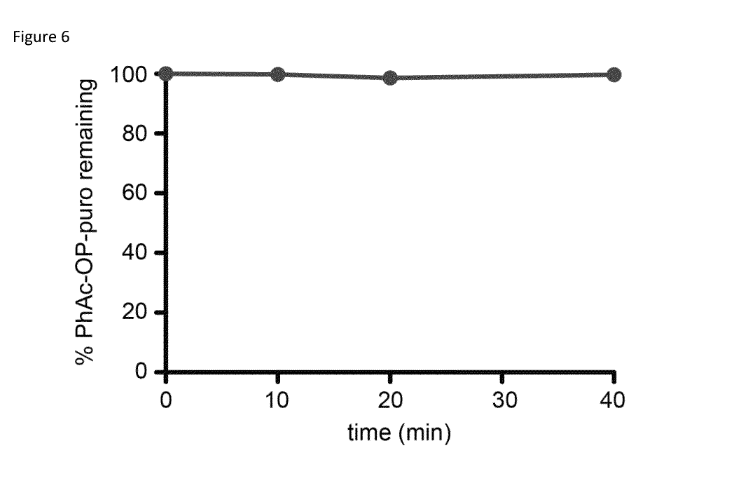

The potential esterase-dependent metabolism of PhAc-OP-puro in mouse plasma was assessed by liquid chromatography mass spectrometry (LCMS). PhAc-OP-puro was determined to be stable in mouse plasma for up to 40 min (FIG. 6). This suggests that PhAc-OP-puro is not a substrate for plasma esterases and that it should be stable in mouse plasma in vivo.

### Example 5

### Testing of PhAc-OP-Puro in Wild Type Mice

Experiments in intact animals to determine the stability of PhAc-OP-puro in vivo can be performed. First, it can be confirmed that labeling of newly synthesized proteins with PhAc-OP-puro treatment is not detected in cells in wild type animals lacking PGA expression. OP-puro can be used as a positive control because previous studies demonstrated that OP-puro could detect newly synthesized proteins in mice (Liu et al 2012 supra). PhAc-OP-puro or OP-puro can be administered intraperitoneally (i.p.) to wild type mice and after a period of time (for example, 30 minutes) the mice can be sacrificed and the labeling of newly synthesized proteins in different tissues (e.g. small intestine, muscle, kidney) can be detected by fluorescence microscopy using click chemistry with Alexa Fluor-568 azide as described above. No OP-puro protein labeling should be detected in wild type mice receiving PhAc-OP-puro.

If proteins are labeled in wild type mice administered PhAc-OP-puro, then it would be likely that PhAc-OP-puro is converted to OP-puro somewhere prior to entering the cells. One possibility is that the conversion is occurring during first-pass metabolism in the liver. To address this potential problem, PhAc-OP-puro can be administered intravenously. Such administration avoids hepatic metabolism.

### Example 6

### Generation of Transgenic Mice that Express PGA in Pancreatic Beta Cells

The Cre-loxP recombination system can be used to generate mice that express PGA specifically in pancreatic beta cells. Initially, a first transgenic mouse line that incorporates a conditional PGA and IRES GFP expression cassette into the commonly targeted and innocuous ROSA26 locus will be generated. ROSA26 will be selected because targeting the transgene there is likely to prevent genetic perturbation by the transgene construct (Zambrowicz B P et al, Proc Nati Acad Sci USA 94, 3789 (1997); incorporated by reference herein). The CAG promoter, which contains the cytomegalovirus early enhancer and the chicken beta-actin promoter will be selected because it is ubiquitously active in mammalian cells and drives high levels of gene expression (Alexopoulou A N et al, BMC Cell Biol 9, 2 (2008); incorporated by reference herein).

The construction of this expression module will be done using standard embryonic stem cell targeting techniques followed by selection and confirmation of proper insertion using a combination of PCR screening and Southern blotting. Confirmed stem cells will be injected into blastocytes to generate founder chimeric mice. Germ-line transmission of transgene founders will be identified using techniques known in the art.

Once generated, these conditional PGA transgenic mice (floxed STOP PGA mice) can be crossed to mice that express Cre specifically in pancreatic beta cells (strain: B6.Cg-Tg(Ins2-cre)25Mgn/J). Specific expression of PGA in pancreatic beta cells in PGA-Inst-Cre mice can be confirmed using immunocytochemistry with an antibody to GFP as described above using cultured cells (shown in FIG. 5).

### Example 7

### Translational Regulation in Pancreatic Beta Cells

One example of how the disclosed compositions and methods can be used is the study of translational control of glucose signaling in the pancreas. The disclosed methods also can be used to perform cell-specific, nascent proteome profiling in the tissues of any multicellular organism.

Metabolic homeostasis is controlled by tightly regulating blood glucose levels (Gomez E et al, J Biol Chem 279, 53937 (2004); Triplitt C L, Am J Manag Care 18, S4 (2012); both of which are incorporated by reference herein). An increase in blood glucose causes release of insulin from beta cells; conversely, low glucose levels stimulate the release of glucagon from adjacent alpha cells. Insulin causes glucose uptake into cells, thereby reducing blood glucose concentrations. Defects in insulin signaling or the loss of beta cells results in hyperglycemia, the signature of diabetes. Elevations in glucagon levels add to abnormal glucose handling (Christensen M et al, Rev Diabet Stud 8, 369 (2011); incorporated by reference herein). In the mouse, glucagon-producing alpha cells form a rim around the outer edge of the islet with insulin-producing beta cells residing in the center; in humans, these two types of cells are more interspersed (Kulkarni R N, Intl Biochem Cell Biol 36, 365, (2004); incorporated by reference herein). In both instances, however, cellular behavior is strongly influenced by context, such that critical regulatory influences are lost when the islet architecture is disrupted (Yang Y P et al, Gene Dev 25, 1680 (2011); incorporated by reference herein).

Glucose can rapidly stimulate not only the translation of proinsulin (Itoh N et al, FEBS Lett 93, 343 (1978) and Itoh N et al, Nature 283, 100 (1980); both of which are incorporated by reference herein) but also translation of the prohormone convertases PC2 and PC3, enzymes required for processing of the insulin precursor (Alarcon C et al, J Biol Chem 268, 4276 (1993); incorporated by reference herein). Possibly, other components, both known and unknown, of the insulin secretory pathway could be regulated at this level as well. The set of beta cell proteins regulated by glucose at the translational level has never been identified.

Although it might be possible to gain insights into these translationally regulated products by examining isolated cells, it is likely that essential regulatory components will depend on paracrine signals, growth factors, and gap junction interactions that depend upon islet integrity. Once the family of translationally-regulated proteins is identified, it should be possible to determine whether a single pathway or multiple pathways coordinate the translational effects. Recent advances in understanding the mechanisms of translational enhancement (RNA binding proteins, microRNA accessibility, etc.) make this a potentially fruitful area for study. Identifying the glucose-stimulated nascent proteome in beta cells in animals thus would provide insight into beta cell function in a physiologically relevant context and could potentially reveal strategies to enhance beta cell function in diabetes.

Although glucose rapidly induces protein synthesis in pancreatic beta cells, the entire set of glucose-regulated proteins has not been identified. To address the question of how protein translation is regulated by glucose in pancreatic beta cells, the disclosed methods can be used as follows.

The PGA-Ins2-Cre mice generated as described above will be used to determine whether or not newly synthesized proteins can be detected upon PhAc-PO-puro treatment specifically in pancreatic beta cells expressing PGA in isolated islets. Initial experiments will be conducted using isolated pancreatic islets from the transgenic mice. Isolation of pancreatic islets is described in the art (Itoh N et at 1980 supra). Pancreatic islets expressing PGA can be treated with glucose followed by PhAc-OP-puro (5 μM) for 30 minutes, which, as shown above, is sufficient time to achieve robust labeling of newly synthesized proteins. Labeling of newly synthesized proteins can be determined by fluorescence microscopy using click chemistry with Alexa Fluor-568 azide as described above. Fluorescence detection of newly synthesized proteins in beta cells (identified using an antibody against insulin, which should co-localize with GFP) but not in alpha cells (identified using an antibody against glucagon) will demonstrate that beta cell-specific labeling of newly synthesized proteins in heterogeneous tissues can be achieved.

It will next be determined whether new protein synthesis can be detected specifically in pancreatic beta cells in intact transgenic mice with beta cell PGA expression. PhAc-OP-puro will be administered i.p. (or i.v., if necessary as described above) into PGA-Ins2-Cre mice. The concentration of PhAc-OP-puro as well as the time necessary to detect robust labeling of newly synthesized proteins in PGA-expressing beta cells in animals can be optimized using standard techniques. Labeling of newly synthesized proteins will be determined by fluorescence microscopy using click chemistry with Alexa Fluor-568 azide as described above after harvesting of pancreatic islets.

Once an optimal concentration and time has been determined, PhAc-OP-puro can be administered floxed STOP PGA mice that were not crossed with Ins2-Cre mice as a negative control. There should not be any labeling of newly synthesized proteins in pancreatic beta cells in these mice. Together, these experiments will determine whether selective labeling of newly synthesized proteins can be identified in pancreatic beta cells in isolated pancreatic islets from PGA-Ins2-Cre mice and in vivo in PGA-Ins2-Cre mice.

The glucose-stimulated proteome in pancreatic beta cells in PGA-Ins2-Cre mice can then be profiled. PGA-Ins2-Cre Mice can be given an i.p. glucose challenge, which has been shown to increase blood glucose levels and insulin release 30 minutes after injection (Gustavsson N et al, Proc Natl Acad Sci USA 105, 3992 (2008); incorporated by reference herein), followed by treatment (intraperitoneally or intravenously if necessary) with PhAc-OP-puro or vehicle control.

After the previously determined optimal time for PhAc-OP-puro labeling, the mice will be sacrificed and the pancreatic islets will be isolated and homogenized. For initial studies five mice per treatment condition will be pooled in order to obtain enough material for MS/MS analysis. This number of mice was selected based upon the findings using Hela cells (above) and the approximate number of beta cells in the pancreatic islet.

Pancreatic islet homogenates will be incubated with biotin azide under the click reaction conditions. Biotinylated proteins will be affinity purified with NeutrAvidin agarose beads. The captured biotinylated proteins from both treatment conditions will be alkylated and digested on the agarose beads using trypsin. The resultant peptides will be subjected to mass spectrometry to identify the proteins induced in response to glucose.

1. A compound with the formula: 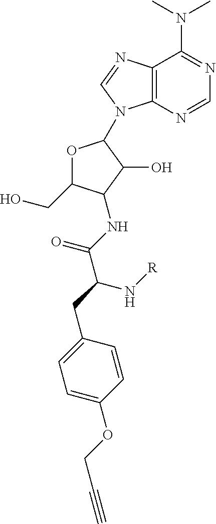
wherein R is acyl.

2. The compound of claim 1 with the structure:

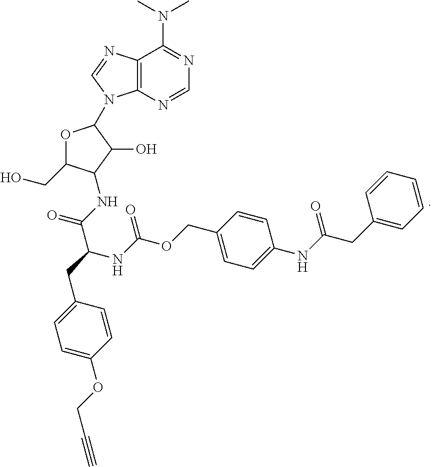 [SMILES: C#CCOc1ccc(C[C@H](NC(=O)OCc2ccc(NC(=O)Cc3ccccc3)cc2)C(=O)NC2C(CO)OC(n3cnc4c(N(C)C)ncnc43)C2O)cc1]

3. A method of generating an isolated set of proteins from a subject, the method comprising:
administering a blocked puromycin analog to the subject, wherein the blocked puromycin analog can be converted into an active puromycin analog by a penicillin acylase and wherein the subject is a transgenic multicellular organism that expresses the penicillin acylase in a selected cell type and wherein the active puromycin analog is conjugated to the set of proteins in the selected cell type during translation resulting in a conjugated puromycin analog;
obtaining a sample from the subject, the sample comprising cells of the selected cell type;
purifying the set of proteins from the sample on the basis of the conjugated puromycin analog, thereby generating an isolated set of proteins.

4. The method of claim 3 wherein the blocked puromycin analog comprises the compound of claim 1 and wherein the active puromycin analog is O-propargyl puromycin.

5. The method of claim 4 wherein the penicillin acylase is penicillin G acylase.

6. The method of claim 5 wherein the penicillin G acylase comprises a polypeptide of SEQ ID NO: 1, SEQ ID NO: 2, SEQ ID NO: 3, or SEQ ID NO: 4, or a homolog with at least 90% identity thereto, provided that said homolog can conjugate a blocked puromycin analog to the set of proteins.

7. The method of claim 3 further comprising adding a label to the set of proteins wherein the label is conjugated to the protein via the conjugated puromycin analog.

8. The method of claim 7 wherein adding the label is performed using click chemistry with a fluorescent azide.

9. The method of claim 8 wherein the label comprises a fluorophore or biotin.

10. The method of claim 3 wherein the subject comprises a first nucleic acid construct comprising a first sequence that encodes a penicillin G acylase, a second sequence comprising a loxP-flanked STOP cassette, wherein the loxP-flanked STOP cassette prevents expression of the penicillin G acylase; and a third sequence comprising a constitutively active promoter, wherein the constitutively active promoter is operably linked to the first sequence and the second sequence and a second nucleic acid construct, the second nucleic acid construct comprising a fourth nucleic acid sequence that encodes a cre recombinase and a fifth nucleic acid sequence that comprises a conditionally active promoter, wherein the conditionally active promoter is operably linked to the fourth nucleic acid sequence.

11. The method of claim 10 wherein the conditionally active promoter is a tissue specific promoter or a cell specific promoter.

12. The method of claim 3 wherein the subject is a mouse or rat.

13. The method of claim 12 wherein the selected cell type is pancreatic beta cells and wherein the conditionally active promoter is a pancreatic beta cell specific promoter.

14. The method of claim 3 further comprising performing mass spectrometry analysis on the isolated set of proteins.

15. A transgenic mouse comprising
a first nucleic acid construct, the first nucleic acid construct comprising a first sequence encoding an acylase, a second sequence comprising a loxP-flanked STOP cassette that prevents the expression of the acylase, and a third sequence comprising a constitutively active promoter, wherein the constitutively active promoter is operably linked to the penicillin acylase.

16. The mouse of claim 15 wherein the penicillin acylase comprises a penicillin G acylase.

17. The mouse of claim 16 wherein the penicillin G acylase comprises a polypeptide of SEQ ID NO: 1, SEQ ID NO: 2, SEQ ID NO: 3, or SEQ ID NO: 4, or a homolog with at least 90% identity thereto provided that said homolog can conjugate a blocked puromycin analog to a set of proteins in the mouse.

18. The transgenic mouse of claim 14 further comprising:
A second nucleic acid construct, the second nucleic acid construct comprising a first sequence that encodes a cre recombinase and a second sequence that comprises a conditionally active promoter.

19. The transgenic mouse of claim 18 wherein the conditionally active promoter is a tissue specific or cell specific promoter.


SEQUENCE LISTING (4 sequences)

SEQ ID NO: 1 - protein, 846 aa, Escherichia coli
MKNRNRMIVNCVTASLMYYWSLPALAEQSSSEIKIVRDEYGMPHIYANDTWHLFYGYGYVVAQDRLFQMEMARRSTQGTVAEVLGKDFVKFDKDIRRNYWPDAIRAQIAALSPEDMSILQGYADGMNAWTDKVNTNPETLLPKQFNTFGFTPKRWEPFDVAMIFVGTMANRFSDSTSEIDNLALLTALKDKYGVSQGMAVFNQLKWLVNPSAPTTIAVQESNYPLKFNQQNSQTAALLPRYDLPAPMLDRPAKGADGALLALTAGKNRETIAAQFAQGGANGLAGYPTTSNMWVIGKSKAQDAKAIMVNGPQFGWYAPAYTYGIGLHGAGYDVTGNTPFAYPGLVFGHNGVISWGSTAGFGDDVDIFAERLSAEKPGYYLHNGKWVKMLSREETITVKNGQAETFTVWRTVHGNILQTDQTTQTAYAKSRAWDGKEVASLLAWTHQMKAKNWQEWTQQAAKQALTINWYYADVNGNIGYVHTGAYPDRQSGHDPRLPVPGTGKWDWKGLLPFEMNPKVYNPQSGYIANWNNSPQKDYPASDLFAFLWGGADRVTEIDRLLEQKPRLTADQAWDVIRQTSRQDLNLRLFLPTLQAATSGLTQSDPRRQLVETLTRWDGINLLNDDGKTWQQPPSAILNVWLTSMLKRTVVAAVPMPFDKWYSASGYETTQDGPTGSLNISVGAKILYEAVQGDKSPIPQAVDLFAGKPQQEVVLAALEDTWETLSKRYGNNVSNWKTPAMALTFRANNFFGVPQAAAEETRHQAEYQNRGTENDMIVFSPTTSDRPVLAWDVVAPGQSGFIAPDGTVDKHYEDQLKMYENFGRKSLWLTKQDVEAHKESQEVLHVQR

SEQ ID NO: 2 - protein, 843 aa, Achromobaceter xylosoxidans
MAQPVAQAAGQESAAVAAPAQGAAGKVTIRRDAHGMPHVYADTVYGIFYGYGYAVAQDRLFQMEMARRSTQGRVAEVLGASMVAFDKSIRGNFSPERIQRQLAALPAADRQVLDGYAAGMNAWLARIRAQPGQLMPKEFNDLGFAPADWTAYDVAMIFIGTMANRFSDANSEIDNLALLTALKDRHGEAEAMRIFNQLRWLTDSRAPTTVPPQEGSYQPAVFQPEGAEQLAYALPRYDGTPPMLERVVRDPATRGVVDGAPATLRARLAEQYAQSGQPGIAGFPTTSNMWIVGRDHAKDARSILLNGPQFGWWNPAYTYGIGLHGAGFDVVGNTPFAYPSILFGHNAHVAWGSTAGFGDDVDIYAEKLDPADRNRYFHDGQWKTLEKRTDLILVKDAAPVTLDVYRSVHGLIVKFDDAQHVAYAKARAWEGFELQSLMAWTRKTQSANWEQWKTQAARHALTINWYYADDRGNIGYAHTGFYPRRRPGHDPRLPVPGTGEMDWLGLLPFSTNPQVYNPGQGFIANWNNQPMRGYPSTDLFAIVWGQADRYAEIETRLKAMTANGGKVSPQQMWDLIRTTSYADVNRRHFLPFLQRAVQGLPADDPRVRLVAGLGGWDGMMTSEREPGYYDNAGPAVMDAWLRAMLKRTLADEMPADFFKWYSATGYPTPQAPATGSLNLTTGVKVLFNALAGPAAGVPQRYDFFNGARADDVILAALDDALAALRQAYGKDPAAWKIPAPPMVFAPKNFLGVPQADAKAVLSYPATQNRGTENNMTVFDGKSVRAVDVVAPGQSGFVAPDGTPSPHTRDQFDLYNSFGSKRVWFTADEVRRNATSEETLRYRR

SEQ ID NO: 3 - protein, 816 aa, Alcaligenes faecalis
MQKGLVRTGVAAAGLILGLAWAPAHAQVQSVEVMRDSYGVPHVFADSHYGLYYGYGYAVAQDRLFQMDMARRSFVGTTAAVLGPGEQDVYVKYDMQVRQNFTPASIQRQIAALSKDERDIFRGYADGYNAYLEQVRRRPELLPKEYVDFDFQPEPLTDFDVVMIWVGSMANRFSDTNLEVTALAMRQSLEKQHGPERGRALFDELLWINDTTAPTTVPAPAAERKPQAQAGTQNLAHVSSQVLAAELERQDKHWGGRGPDFAPKASNLWSTRPERVQDGSTVLINGPQFGWYNPAYTYGIGLHGAGFDVVGNTPFAYPIVLFGTNSEIAWGATAGPQDVVDMYQEKLNPARADQYWFNNAWRTMEQRKERIQVRGQADREMTIWRTVHGPVMQFDYEQGTAYSKKRSWDGYEVQSLLAWLNVAKARNWTEFLDQASKMAISINWYYADKHGNIGYVSPAFLPQRPADQDIRVPAKGDGSMVWQGIKSFDAIPKAYNPPQGYLVNWNNKPAPDKTNTDTYYWTYGDRMNELTSQYQDKDLHSVQEIWGFNKKASYSDVNWRYFRPHLEKLAQSLPANDAGQAALTTLLAWDGMEHDQAGQNAGPARVLFKTWLEEMYKQVLMPVVPESHRAMYSQTGFATQQGPNPGSINLSMGTKVLLRALALEAHPDPKRVNVFGERSSQETMHTALQNAQARLSQEQGAQMERWTMPTSVHRFSDKNFTGTPQTTSGNTFAFTGYQNRGTENNRVAFDAKSVEFCDAMPPGQSGFTDRNGVRSPHYEDQLKLYENFECKTMDVTHTDIRRNAQSSTMLLIQPQP

SEQ ID NO: 4 - protein, 805 aa, Bacillis badius
MKKRWLMMIMIVFALVLPQNFTFAKEQKKVENVTIIRDSYGVPHLYAKNKKDLYKAYGYVMAQDRLFQLEMFRRGNEGTVSEIFGEEYVTKDEQSRRDGYSDQEIQKMLNGLDGETKQLIEQFAEGITVYVNEAVKDQDKKLSKEFHDYRFLPRKWKATDVVRLYMVSMTYFMDNHQELKNAEILARLERTYGKEKAAKMFEDLVWKNDLEAPASIQPDDQTEAKKDGKTSIQPLSSAVIDASKKIVKEREEFVQSSEELGLPLKIGSNAMIVGAKKSKSGNALLFSGPQVGFVAPGFLYEVGLHSPGFDMEGSGFIGYPFIMFGANQQLALTATAGYGNVTDIFEERLNPANPAQYFYKGKWRNMEKRTETFIVRGENGKAKKVEKVFFHTVHGPVISMEKEKNVAYSKSWSFRGTGAQSIQAYMKANWAKSLKEFQQAASEFTMSLNWYYADKKGNIAYYHVGKYPIRSNQIDDRFPTPGTGEYEWKGFQSFSKNPQAINPKKGYVVNWNNKPAKYWRNGEYSIVWGKDNRVQQFINGMEARDKVDLNDLNEINYTASFAQLRTHYFKPLLIKTLKKYQSENKEYAYLVEQLQKWNNLKEDKNNDGYYDAGVAAFFDEWWNNAHNKLFNDSLNTVSDLTREITDHRMGATLAYKVLSGEPTNYQWKTKEEAEKIILDSANEALAKLHEEKGTAADQWRMPIKTMTFGAKSLIAIPHGYGSKTEIIEMNRGSENHYIEMTPKQPEGFNVTPPGQIGFIHKDGTLSDHYEDQLSLYANWKFKPFLFDKKDVKRAAVSVSELNTGK